(19) United States
(12) Patent Application Publication
(10) Pub. No.: US 2026/0315138 A1
(43) Pub. Date: Oct. 15, 2026

(54) PRECISIONSAFE ELECTROSURGERY PEN

(71) Applicant: RUTGERS, THE STATE UNIVERSITY OF NEW JERSEY, New Brunswick, NJ (US)

(72) Inventor: Joseph G. Barone, New Brunswick, NJ (US)

(73) Assignee: RUTGERS, THE STATE UNIVERSITY OF NEW JERSEY, New Brunswick, NJ (US)

(21) Appl. No.: 19/644,732

(22) Filed: Apr. 10, 2026

Related U.S. Application Data
(60) Provisional application No. 63/786,493, filed on Apr. 10, 2025.

(51) Int. Cl. A61B 18/14; A61B 18/00

(52) U.S. Cl. CPC A61B 18/1477; A61B 2018/0091; A61B 2018/1425; A61B 2018/1475; A61B 2218/008

(57) ABSTRACT

An electrosurgical pen designed for pediatric surgery comprises input control buttons positioned near the distal tip electrode, as well as one or more proximal limbs configured to provide grip stability and improved precision control. The electrosurgical pen includes safety features, such as a retractable electrode tip. An electrical cable and/or suction tube connected to the proximal end of the electrosurgical pen permit a wide range of motion. A form-fitting holster can be used to magnetically secure the electrosurgical pen and prevent accidental injuries.

### CROSS-REFERENCE TO RELATED APPLICATION

This application claims priority to U.S. Provisional Application No. 63/786,493 that was filed on Apr. 10, 2025. The entire content of the application referenced above is hereby incorporated by reference herein.

### FIELD

The present invention generally relates to electrosurgery instruments, and, more particularly, to electrosurgery pens with improved precision and safety for pediatric surgery.

### BACKGROUND

Electrosurgical pens or pencils are medical devices used to cut, cauterize, and coagulate tissue through the application of high-frequency (e.g., radio-frequency or RF) alternating current, such as described in U.S. Pat. Nos. 8,882,767 and 8,882,768, the disclosures of which are incorporated by reference herein. RF electrosurgery is performed using a RF electrosurgical generator (also referred to as an electrosurgical unit or ESU). Monopolar and/or bipolar methods can be used to conduct RF current from the generator via a cable or cord to the electrode tip of the electrosurgical pen. A surgeon uses the electrode tip of the pen to heat tissue to varying degrees, resulting in ablation, desiccation, coagulation, and/or vaporization.

RF electrosurgery is commonly used in many surgical disciplines, including dermatological, gynecological, cardiac, plastic, ocular, spine, ENT, maxillofacial, orthopedic, urological, neuro- and general surgical procedures, as well as certain dental procedures.

However, there are no electrosurgical pens specifically designed for pediatric surgery, which requires fine movement and precise control for working in small surgical spaces. Currently available electrosurgical pens are designed with built-in control buttons (e.g., for cutting and cauterizing) positioned in the middle of the pen, requiring the surgeon to use his or her shoulder to “write” with the pen. Shoulder writing usually requires a large working area and is prone to lack of control and precision. Furthermore, typical electrosurgical pens that are used for adult surgery utilize blunt tips for safety purposes. For pediatric surgery, needle tips are preferred for more precise cutting; however, needle tips carry an increased risk of injury to surgeons and operating room assistants.

Thus, there is a need for an electrosurgical pen that can be safely and effectively used for pediatric surgery, or for other procedures that are performed within a small surgical field and require the surgeon to have a high level of control, precision, and stability.

### BRIEF SUMMARY

An electrosurgical pen designed for pediatric surgery comprises input control buttons positioned near the distal tip electrode, as well as one or more proximal limbs configured to provide grip stability and improved precision control. The electrosurgical pen includes safety features, such as a retractable electrode tip. An electrical cable and/or suction tube connected to the proximal end of the electrosurgical pen permit a wide range of motion. A form-fitting holster can be used to magnetically secure the electrosurgical pen and prevent accidental injuries.

Certain embodiments provide an electrosurgical instrument comprising: a handpiece comprising a proximal end portion including a proximal end, a distal end portion including a distal end, and a distal electrode tip proximate the distal end; an electrical cable connected to the proximal end and configured to be coupled to a generator; a suction tube connected to the proximal end configured to evacuate smoke or fluid; and an input device positioned on the distal end portion within about 0.61 to about 0.81 inches from the distal end, the input device configured to control operation of the electrosurgical instrument.

In certain embodiments, the proximal end portion comprises at least two limbs, and wherein the at least two limbs are joined together at a crook-shaped joint connected to the distal end portion.

In certain embodiments, the at least two limbs are formed in a Y-shape, U-Shape, or O-shape.

In certain embodiments, the crook-shaped joint and distal end portion are configured to support a user's forefinger.

In certain embodiments, the input device is positioned within about 0.42 to about 0.57 inches from the crook-shaped joint.

In certain embodiments, the at least two limbs form an elliptical or circular shape and join together at a proximal crook-shaped joint connected to a proximal end outlet.

In certain embodiments, the electrical cable and the suction tube travel proximally through the proximal end outlet.

In certain embodiments, the suction tube is configured to bifurcate at the proximal and distal crook-shaped to allow for evacuation of smoke or fluids thorough either or both limbs.

In certain embodiments, the distal end portion is configured to be held by a user's thumb and middle finger.

In certain embodiments, the distal electrode tip is retractable.

In certain embodiments, the distal electrode tip is a needle.

In certain embodiments, the distal electrode tip is about 0.31 to about 1.00 inches in length.

In certain embodiments, the input device comprises at least one control button.

In certain embodiments, the input device is configured to transmit at least one control signal to the generator, the control signal configured to cause the generator to deliver electrical energy to the distal electrode tip.

In certain embodiments, the length of the electrosurgical instrument, including the distal electrode tip, is about 4.70 to about 5.40 inches.

In certain embodiments, the electrosurgical instrument further comprises a recess including a first magnet.

In certain embodiments, the recess is located on a back-side of the distal end portion.

In certain embodiments, the first magnet is configured to attract a second magnet located inside a form-fitting holster, the holster configured to hold the electrosurgical instrument when not in use.

In certain embodiments, the distal end portion comprises one or more fume extraction ports.

In certain embodiments, the electrosurgical instrument further comprises a fume extraction tube extending distally from the distal end portion.

Other objects, features, and advantages of the present invention will be apparent to one of skill in the art from the following detailed description and figures.

### BRIEF DESCRIPTION OF THE FIGURES

The present application can be understood by reference to the following drawings, wherein like reference numerals represent like elements. The drawings are merely exemplary to illustrate certain features that may be used singularly or in combination with other features and the present application should not be limited to the embodiments shown.

FIG. 1A is a front view of an embodiment of an electrosurgical pen in accordance with the present invention.

FIG. 1B is a front, side perspective view of the electrosurgical pen of FIG. 1A.

FIG. 1C is a side view of the electrosurgical pen of FIG. 1A.

FIG. 2 is a photograph illustrating an exemplary embodiment of a three-dimensional model of an electrosurgical pen in accordance with the present invention.

FIG. 3 is a photograph illustrating an exemplary embodiment of a three-dimensional model of a holster for the electrosurgical pen of FIG. 2.

FIG. 4 is a photograph illustrating an exemplary manner of holding an electrosurgical pen in accordance with the present invention.

FIGS. 5A-5D are top perspective views of an embodiment of an electrosurgical pen in accordance with the present invention, showing fume extraction ports (5A), a fume extraction tube (5B), an internal control board associated with the control buttons (5C), and internal wiring (5D).

FIGS. 6A-6B are top perspective views of an embodiment of an electrosurgical pen in accordance with the present invention, showing convergence of the proximal ends into a single outlet (6A) that can serve as a conduit for both electrical wiring and suction tubing (6B).

FIG. 7 is a drawing illustrating an exemplary manner of holding an electrosurgical pen in accordance with the present invention.

### DETAILED DESCRIPTION

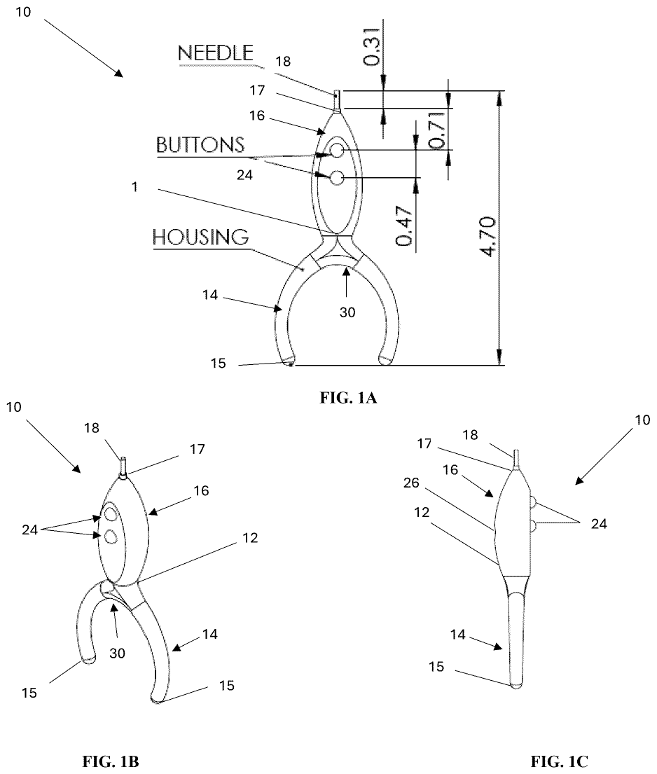

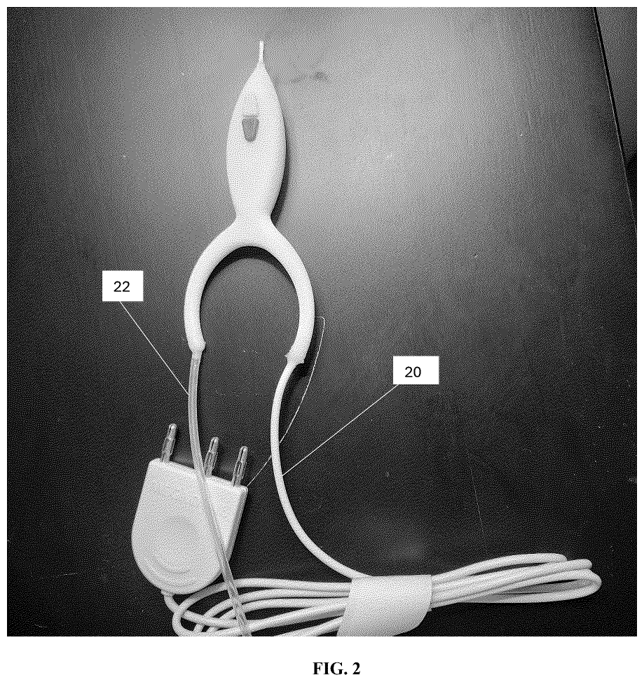

Referring now to the drawings wherein like reference numerals are used to identify like elements in the various views, FIGS. 1A-1C illustrate an exemplary embodiment of an electrosurgical pen 10 designed for pediatric surgery or procedures performed within a small surgical field. The electrosurgical pen 10 includes housing comprising a handpiece 12 having a proximal end portion 14, including a proximal end 15, and a distal end portion 16, including a distal end 17. A retractable electrode tip (also referred to as a retractable needle) 18 is proximate the distal end 17.

As shown in FIG. 2, an electrical connector or cable 20 is attached to the electrosurgical pen 10 at the proximal end 15. The cable 20 can be plugged into an electrosurgical generator (not shown) to communicate electrical energy from the generator to the electrosurgical pen 10.

The electrical energy travels through the pen's retractable electrode tip 18 to a patient's tissue.

The cable 20 can also include additional conductors, such as for transmitting control signals from the electrosurgical pen 10 (e.g., control buttons 24, described below with respect to FIGS. 1A-1C) to the electrosurgical generator. The control signals can be used to cause the generator to deliver RF currents to the electrosurgical pen 10 for different cutting modes, such as cut, cauterize, coagulate, and cut-coagulate blend.

As further shown in FIG. 2, a suction tube 22 is also attached to the electrosurgical pen 10 at the proximal end 15. The suction tube 22 can be used to suction smoke created during electrocautery or fluid (e.g., blood) from the surgical field, and it connects to a small hole (not shown) at the proximal end 15 of the electrosurgical pen 10. In comparison to currently available electrosurgical pens, the suction tube 22 is less bulky and stiff, allowing for a greater range of motion.

The exemplary electrosurgical pen 10 further includes control buttons 24, shown in FIGS. 1A-1C, that serve as input devices providing various functions, such as cutting and cauterizing. The control buttons 24 can be similar to those of existing electrosurgical pens (e.g., in color and positioning with respect to one another), so as to maintain familiarity for the operating surgeon. While two control buttons 24 are shown in FIGS. 1A-1C, it should be noted that an electrosurgical pen in accordance with the present disclosure can include one control button or more than two control buttons (e.g., three, four, or five or more control buttons), and the control buttons can vary in shape, size, color, and position.

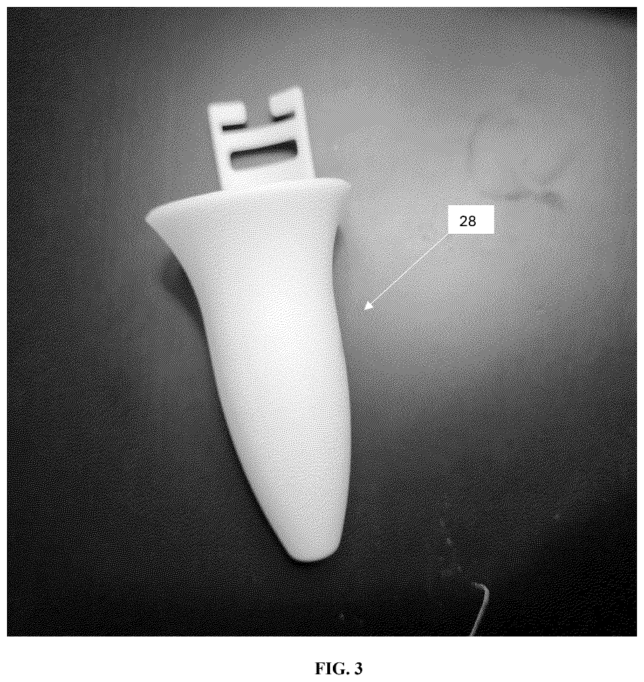

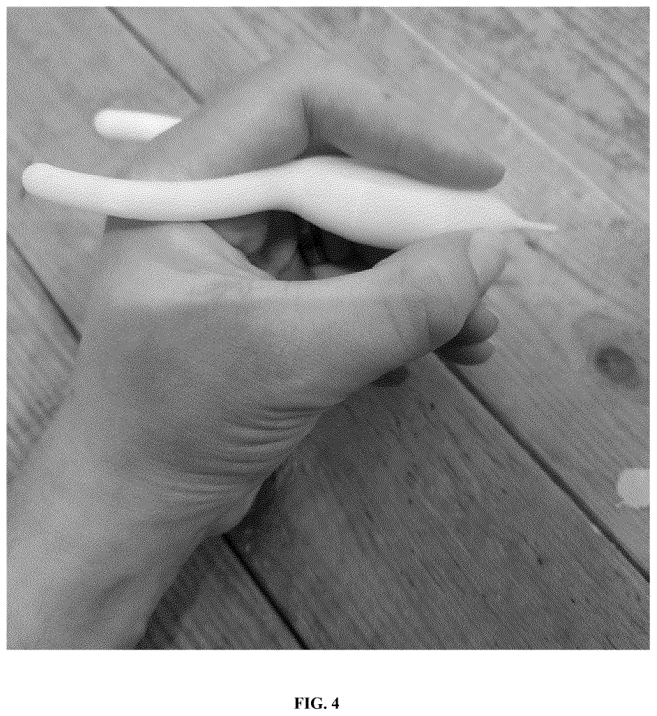

A portion of the handpiece 12, such as a back-side recess 26 (shown in FIG. 1C), can include or be attached to a magnet (not shown), which can be configured to couple with (e.g., be electromagnetically attracted to) a second magnet inside of a form-fitting holster 28 (shown in FIG. 3) for the electrosurgical pen 10. The magnets on the handpiece 12 and inside the holster 28 help to secure the electrosurgical pen 10 inside the holster 28 and prevent it from falling out and potentially causing injury (e.g., from the needle tip) to the surgeon or operating assistants. In combination with the retractable feature of the needle, the magnets securing the electromagnetic pen 10 to the holster 28 can provide enhanced safety in comparison to existing electrosurgical pens.

Referring now to FIG. 4, additional features and advantages of the disclosed electrosurgical pen 10 can be seen. As suggested by the name, an electrosurgical pen is held in the same manner as a pen or pencil when writing. In contrast to existing electrosurgical pens that are elongated (e.g., similar to a typical pen in length), the disclosed electrosurgical pen 10 is more compact, shorter in length, and has a Y-shaped design. Other designs, such as a U-shape or O-shape, for example, can also be used. As shown in FIG. 4, a surgeon's forefinger can rest on the distal end portion 16 of the handpiece 12, near or on top of the control buttons 24, while the distal end portion 16 of the handpiece 12 is also held by the surgeon's thumb and middle finger. The Y-shape of the proximal end portion 14 of the electrosurgical pen 10 provides a crook-shaped joint section 30, between the two “limbs” of the proximal housing of the handpiece 12, through which the surgeon can position his or her forefinger, as illustrated in FIG. 4 (as noted above, other shapes could provide a similar crook or hole, such as a U-shape or an O-shape). This design of the electrosurgical pen 10, including the crook-shaped joint section 30 and distal housing upon which the surgeon can rest his or her forefinger, provides added stability (e.g., in comparison to a typical elongated pen-shaped device). It should also be noted that while the distal end portion 16 of the handpiece 12 is elliptically-shaped in FIGS. 1, 2, and 4, it can be any shape, including a cylinder or square-shape, for example.

Returning to FIG. 1A, exemplary dimensions of the electrosurgical pen 10 are shown. In an example, the length of the electrosurgical pen 10 from the proximal end 15 to the distal electrode tip 18 can be about 4.70 to about 5.40 inches. In the specific example shown in FIG. 1A, the length of the electrosurgical pen 10 from the proximal end 15 to the distal electrode tip 18 is about 4.70 inches. In an example, the distal electrode tip can be about 0.31 to about 1.00 inches when not retracted. In the specific example shown in FIG. 1A, the distal electrode tip is about 0.31 inches in length. In an example, the distance from the distal-most button 24 to the distal end 17 can be about 0.61 to about 0.81 inches. In the specific example shown in FIG. 1A, the distance from the distal-most button 24 to the distal end 17 is about 0.71 inches. In an example, the distance from the crook-shaped joint section 30 of the Y-shaped handpiece to the proximal-most button 24 can be about 0.42 to about 0.57 inches. In the specific example shown in FIG. 1A, the distance from the crook-shaped joint section 30 of the Y-shaped handpiece to the proximal-most button 24 is about 0.47 inches. It should be noted that all of the above measurements are only examples and other dimensions could be used in accordance with the presently disclosed invention.

Importantly, the control buttons 24 of the electrosurgical pen 10 are positioned closer to the distal end 17 and distal electrode tip 18 than the buttons of typical electrosurgical pens. This permits the operating surgeon to “write” with his or her wrist, rather than with his or her shoulder. Wrist writing allows for more precision and accuracy, finer detail, and improved control in smaller operating fields, such as those that are often encountered while operating on pediatric patients.

Thus, in comparison to existing electrosurgical pens, the presently disclosed electrosurgical pen 10 provides a more compact design with control buttons positioned closer to the distal end, thereby permitting increased precision and control; a Y-shaped design providing increased stability; less bulky electrical and suction tubing for increased range of motion; and a retractable tip and magnetic features providing improved safety.

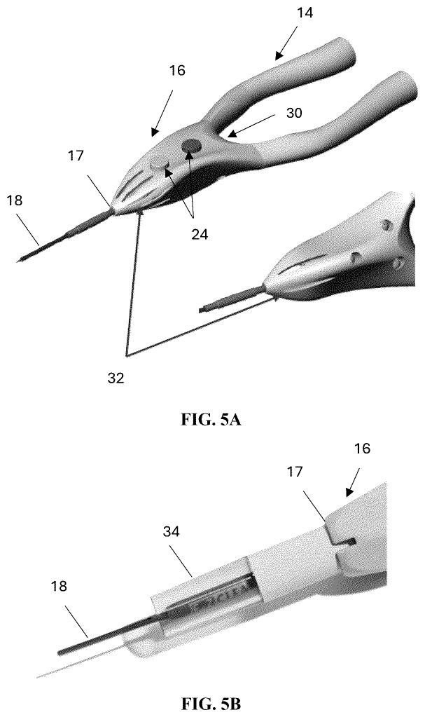

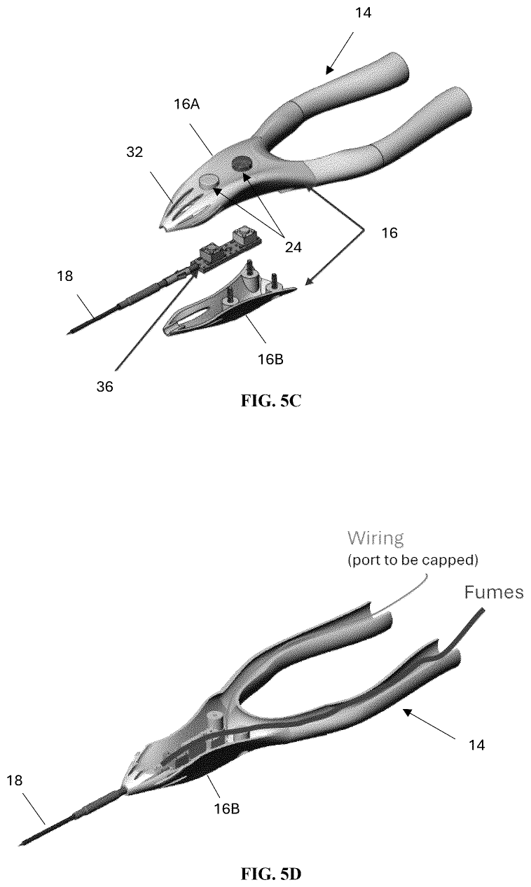

Referring now to FIGS. 5A-5D, another embodiment of the disclosed electrosurgical pen 10 is illustrated. As shown in FIG. 5A, the distal end portion 16 includes one or more fume extraction ports 32 located between the distal end 17 and the control buttons 24. The fume extraction ports 32 are configured to allow fumes generated during electrosurgery to escape at the distal end of the device, in addition to being evacuated via the suction tube 22 at the proximal end of the device (as shown, for example, in FIG. 2). Alternatively, or in addition to the fume extraction ports 32, the electrosurgical pen 10 can include a fume extraction tube 34 extending from the distal end 17 of the device, as shown in FIG. 5B. The fume extraction tube 34 may surround a portion of the retractable electrode tip 18, as shown.

FIG. 5C includes a partially exploded view of the distal end portion 16 of the device, showing the top piece 16A and the bottom piece 16B. A control board 36 is sandwiched between the top piece 16A and bottom piece 16B. The control board is coupled to both the control buttons 24 and the electrode tip 18 via wiring, allowing an operator of the device to use the control buttons 24 to control the function of the electrode tip 18. FIG. 5D is a longitudinal cross-sectional view of the device showing the inner components and bottom housing of the proximal end portion 14. Wiring extending from the control board 36 runs through one limb of the proximal end portion 14 and out through the proximal end 15 (such as via cable 20, as shown in FIG. 2), while smoke fumes or other fluids generated during electrosurgery run through the other limb of the proximal end portion 14 from the electrode tip 18 to the proximal end 15 (such as via suction tube 22, as shown in FIG. 2).

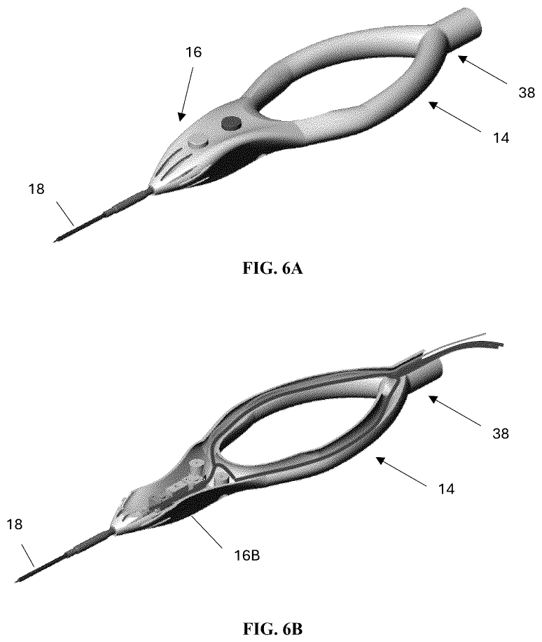

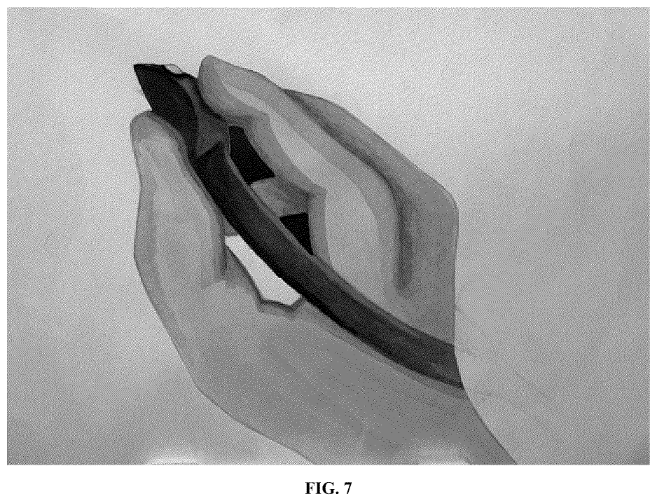

Turning now to FIG. 6A, yet another embodiment of the disclosed electrosurgical pen 10 is illustrated. This embodiment is similar to the one shown and described above with respect to FIGS. 5A-5D, with one important distinction: the limbs of the proximal end portion 14 converge into a single proximal end outlet 38 (as opposed to two separate proximal ends 15). As shown in FIG. 6B, the proximal end outlet 38 allows for convergence of the wiring and exhaust fumes or fluid such that they travel in a single conduit and exit the device through a proximal end port (not shown). It should be noted that the fumes/fluid can travel proximally from the electrode tip 18 through either limb of the of the proximal end portion 14, which allows for more efficient collection of the fumes/fluid. Further, it is believed that the proximal end outlet 38 provides a more balanced design (e.g., in comparison to the “fork” design with two separate proximal ends 15), due to the centralized collection port including both wiring and exhaust fumes/fluid. The balanced design and manner in which a surgeon can hold the single-outlet embodiment of the electrosurgical pen (e.g., as one would hold a pen or other writing instrument) are illustrated in FIG. 7.

Although at least one embodiment of an electrosurgical pen has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment,” or the like, means that a particular feature, structure, or characteristic described in connection with the embodiment is included in at least one embodiment. Thus, appearances of the phrases “in various embodiments,” “in some embodiments,” “in one embodiment,” or “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The terms “about” and “approximately” may be used throughout the specification when referring to a measurable value, such as an amount, a distance, a temporal duration, and the like. The terms “about” and “approximately” are meant to encompass variations of ±20% or ±10%, in certain embodiments ±5%, in certain embodiments ±1%, in certain embodiments ±0.1% from the specified value, as such variations are appropriate in accordance with the present disclosure. Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An electrosurgical instrument comprising:
a handpiece comprising a proximal end portion including a proximal end, a distal end portion including a distal end, and a distal electrode tip proximate the distal end;
an electrical cable connected to the proximal end and configured to be coupled to a generator;
a suction tube connected to the proximal end configured to evacuate smoke or fluid; and
an input device positioned on the distal end portion within about 0.61 to about 0.81 inches from the distal end, the input device configured to control operation of the electrosurgical instrument.

2. The electrosurgical instrument of claim 1, wherein the proximal end portion comprises at least two limbs, and wherein the at least two limbs are joined together at a distal crook-shaped joint connected to the distal end portion.

3. The electrosurgical instrument of claim 2, wherein the at least two limbs are formed in a Y-shape, U-Shape, or O-shape.

4. The electrosurgical instrument of claim 2, wherein the distal crook-shaped joint and distal end portion are configured to support a user's forefinger.

5. The electrosurgical instrument of claim 2, wherein the input device is positioned within about 0.42 to about 0.57 inches from the distal crook-shaped joint.

6. The electrosurgical instrument of claim 2, the at least two limbs form an elliptical or circular shape and join together at a proximal crook-shaped joint connected to a proximal end outlet.

7. The electrosurgical instrument of claim 6, wherein the electrical cable and the suction tube travel proximally through the proximal end outlet.

8. The electrosurgical instrument of claim 6, wherein the suction tube is configured to bifurcate at the proximal and distal crook-shaped to allow for evacuation of smoke or fluids thorough either or both limbs.

9. The electrosurgical instrument of claim 1, wherein the distal end portion is configured to be held by a user's thumb and middle finger.

10. The electrosurgical instrument of claim 1, wherein the distal electrode tip is retractable.

11. The electrosurgical instrument of claim 1, wherein the distal electrode tip is a needle.

12. The electrosurgical instrument of claim 1, wherein the distal electrode tip is about 0.31 to about 1.00 inches in length.

13. The electrosurgical instrument of claim 1, wherein the input device comprises at least one control button.

14. The electrosurgical instrument of claim 1, wherein the input device is configured to transmit at least one control signal to the generator, the control signal configured to cause the generator to deliver electrical energy to the distal electrode tip.

15. The electrosurgical instrument of claim 1, wherein the length of the electrosurgical instrument, including the distal electrode tip, is about 4.70 to about 5.40 inches.

16. The electrosurgical instrument of claim 1, further comprising a recess including a first magnet.

17. The electrosurgical instrument of claim 16, wherein the recess is located on a back-side of the distal end portion.

18. The electrosurgical instrument of claim 16, wherein the first magnet is configured to attract a second magnet located inside a form-fitting holster, the holster configured to hold the electrosurgical instrument when not in use.

19. The electrosurgical instrument of claim 1, wherein the distal end portion comprises one or more fume extraction ports.

20. The electrosurgical instrument of claim 1, further comprising a fume extraction tube extending distally from the distal end portion.
